(19) United States
(12) Patent Application Publication
(10) Pub. No.: US 2026/0365733 A1
(43) Pub. Date: Nov. 19, 2026

(54) SYNERGY BETWEEN IFNa AND TLR3 LIGANDS IN SENSITIZING PD1-RESISTANT COLD TUMORS TO THERAPEUTIC EFFECTS OF PD-1 BLOCKADE

(71) Applicant: Health Research, Inc., Buffalo, NY (US)

(72) Inventors: Pawel KALINSKI, East Aurora, NY (US); Kathleen KOKOLUS, Amherst, NY (US); Shipra GANDHI, Williamsville, NY (US)

(21) Appl. No.: 19/488,230

(22) PCT Filed: Jun. 3, 2024
(86) PCT No.: PCT/US2024/032210

§ 371 Date: Nov. 26, 2025

Related U.S. Application Data
(60) Provisional application No. 63/471,175, filed on Jun. 5, 2023.

(51) Int. Cl. C07K 16/28; A61K 31/7072; A61K 38/21; A61K 39/395; A61P 35/00

(52) U.S. Cl. CPC C07K 16/2818; A61K 31/7072; A61K 38/212; A61K 39/39541; A61P 35/00

(57) ABSTRACT

Provided is a method for treating an individual for a programmed-death 1(PD-1 ) immune checkpoint therapy resistant cancer. The method involves administering to the individual a combination of a Toll-like receptor 3 (TLR3) agonist and interferon-alpha (IFNα), followed by close in time administration of an anti-PD-1 immunotherapy.

### CROSS REFERENCE TO RELATED APPLICATIONS

The present application claims the benefit of U.S. Provisional Patent Application No. 63/471,175, filed Jun. 5, 2023, the entire disclosure of which is incorporated herein by reference.

### STATEMENT REGARDING FEDERALLY SPONSORED RESEARCH

This invention was made with government support under grant no. 1P01CA234212 awarded by the National Institutes of Health. The government has certain rights in the invention.

### BACKGROUND

Immune checkpoint inhibitors (ICIs) which block the interaction of T cell-expressed checkpoint molecules, such as programmed-death 1 (PD-1), with their ligands within tumor tissues (such as PD-L1 and PD-L2), have become standard of treatment in many advanced cancers and significantly extended survival in patients with previously hard-to-treat malignancies¹⁻³, including metastatic, microsatellite instability—high/mismatch repair—deficient (MSI-H/dMMR) colorectal cancer (CRC)⁴. Since ICIs reactivate the immune system to specifically destroy cancer cells and promote long-lasting immune memory, responders often achieve durable tumor control⁵. However, the therapeutic benefit of ICIs is currently limited by the less than 25% of patients responding overall^{6, 7}. Three major factors have been shown to predict the responding patients: high tumor mutational burden (TMB)⁸⁻¹⁰, presence of CD8+tumor-infiltrating lymphocytes (TILs)¹¹⁻¹⁷ and intratumoral expression of PD-1 ligands^{18, 19}. Although such factors can be interconnected with high TMB load driving Tumor infiltrating lymphocyte TIL infiltration and TIL infiltration promoting IFNγ-driven induction of αPD-L1- and PD-L2 on cancer cells, local stroma, and myeloid cells²⁰, the relation between these factors and their relative values remain controversial²¹⁻²⁶. Interestingly, a recent study demonstrated that artificial increase in TMB is not sufficient to enhance ICI responsiveness of mouse tumors²⁷, raising a possibility that TIL content and PD1 sensitivity may be regulated at least partially independently from the TMB.

Several approaches the increase the numbers of tumor-infiltrating lymphocytes (TILs) have been proposed^{21, 28}. Tumors lacking sufficient cytotoxic T lymphocytes (CTLs) have been classified as “cold” and minimally responsive to immune-based treatment approaches²⁹, thus strategies to increase infiltration of CTLs into the tumor microenvironment (TME) have become increasingly sought. Recently, personalized mRNA vaccines demonstrated a dramatic ability of multi-epitope vaccination to enhance the clinical effectiveness of neoadjuvant PD1 blockade to prevent cancer recurrence³⁰, demonstrating a promise of combinatorial approaches. However, there remains an ongoing and unmet need for combinatorial treatment of cancer, and particularly for treating PD1-resistant “cold tumors”. The present disclosure is pertinent to this need.

### SUMMARY OF THE DISCLOSURE

The disclosure provides an approach to treating immunologically cold tumors. This approach promotes CTL infiltration using a chemokine modulatory (CKM) regimen that induces a pro-immune status in the “cold” TME³¹⁻⁴². CKM combines a type I interferon (IFN), which enhance TLR3 expression with toll-like receptor 3 (TLR3) ligands, such as poly-IC^{34-36, 41} or rintatolimod⁴¹, a selective TLR3 ligand^{43, 44} which does not activate NFKB, thus avoiding tumor necrosis factor alpha (TNFα) induction⁴¹, toxicity^{43, 44} and NFKB/TNFα/PGE2-dependent suppressive aspects of inflammation⁴¹. In addition to reprograming of the stromal and myeloid component of the TME, rintatolimod induces TLR3-driven DC (dendritic cell) maturation and T cell stimulation^{43, 45}, the desirable DC-modulating function functions also amplified by the combination of TLR3 activation and IFNα^{34, 46, 47}. The disclosure accordingly provides a method for treating an individual for a programmed-death 1 (PD-1) immune checkpoint therapy resistant cancer. The method involves administering to the individual a combination of TLR3 agonist and interferon-alpha (IFNα), with or followed by close-in-time administration of an anti-PD-1 immunotherapy.

CKM preferentially activates tumor tissues (which show high baseline activation of NFkB, which is needed to sustain tumor viability, and acts as a co-activator for production of both CTL-attracting and Treg-attracting chemokines)³⁴ and the desirable CTL attraction^{34, 39, 41} ex vivo, without or only marginal activation of healthy tissues, suggesting its potential to achieve local intratumoral effects, even after its systemic application.

In accordance with these in vitro predictions, our recently completed clinical study (NCT03599453) demonstrated sufficient tolerability of systemic CKM and its ability to reprogram the TME for selective CTL influx in triple negative breast cancer patients⁴². Despite these favorable immune effects, the subsequent application of PD1 blockade with an average delay of 1 week after CKM, showed only marginal clinical efficacy⁴², raising the need to evaluate the mechanisms involved in its effectiveness and identify the most effective timing of its application relative to ICI. This disclosure demonstrates that CKM works synergistically with PD-1 blockade, resulting in durable cures in mouse models of CRC, but, in contrast to the expectations resulting from previous work³¹⁻⁴², such synergy requires simultaneous or near simultaneous administration of CKM and anti-PD-1 (αPD1) components. Moreover, the disclosure demonstrates that, in addition to CXCR3- and CCR5-dependent CTL attraction to the TME, the effectiveness CKM+αPD-1 therapy involves conventional dendritic cells (cDC1s) which are involved in the enhancement of systemic anticancer immunity protective of subsequent tumor re-challenge.

### BRIEF DESCRIPTION OF THE FIGURES

For a fuller understanding of the nature and objects of the disclosure, reference should be made to the following detailed description taken in conjunction with the accompanying figures.

FIGS. 1A-1C display graphical representations of CKM inducing expression of CTL-attracting chemokines in vitro and systemic application promotes local expression of CTL-attractants and effector markers in the TME. As shown in FIG. 1A, MC38 cells were treated in culture with 50 μg rintatolimod (Rint), 10,000 IU IFNα (interferon alpha-IFNα), or a combination (CKM). Control cells were cultured in DMEM-based media alone untreated (Untx). After a 24 incubation, cells were harvested and assayed for RNA message by real time quantitative polymerase chain reaction (RTqPCR). Transcripts (normalized to GAPDH) are shown. Statistical significance was determined by one way ANOVA with Tukey's multiple comparisons; **** p<0.0001; N=3/group. As shown in FIG. 1B, mice were inoculated subcutaneously with MC38 cells. On day 17, mice were treated with one dose of CKM (50 μg rintatolimod and 10,000 IU IFNα) injected intraperitoneally (CKM) or not treated (Untx). Three days after treatment, tumors were excised and assayed for RNA message by RT-qPCR. Transcripts (normalized to GAPDH) are shown. Statistical significance was determined by unpaired t test; * p<0.05; N=6-9/group. As shown in FIG. 1C, mice were inoculated subcutaneously with MC38 cells and treated intraperitoneally with PBS, IFNα, rintatolimod (RIN), or CKM with and without the addition of αPD-1. Survival was monitored. Statistical significance was determined by log-rank test; * p<0.05, *** p<0.001; N=15-20/group.

FIGS. 2A-2E display schematics and graphical representation of local and systemic administration of CKM in combination with PD-1 blockade that can abrogate tumor growth in mouse models of colorectal cancer. FIG. 2A displays a schematic of therapeutic schedule. Mice were inoculated with MC38 cells. Concomitant treatment represents three cycles consisting of two doses of CKM (50 μg rintatolimod and 10,000 IU IFNα) one day apart followed by 200 μg αPD-1 the next day. Sequential treatment represents two doses of CKM (50 μg rintatolimod and 10,000 IU IFNα) one day apart directly followed by three doses of αPD-1 (200 μg) one, five, and ten days after the second CKM dose. All treatment was administered intraperitoneally. Controls received intraperitoneal injections of PBS, CKM, or αPD-1 at the same dose or volume as the experimental groups. FIG. 2B displays results of mice inoculated with MC28 tumor cells intraperitoneally. Local treatment of early-stage tumors evaluated using intraperitoneal MC38 tumor and intraperitoneal treatment administration beginning on day 3. FIGS. 2C and 2E display results of systemic treatment of early-stage tumors evaluated using subcutaneous MC38 (FIG. 2C) and CT26 (FIG. 2E) tumors and intraperitoneal treatment administration beginning on day 3. FIG. 2D displays results of systemic treatment of late-stage tumors evaluated using subcutaneous MC38 tumors and intraperitoneal treatment administration beginning on day 8. As shown in FIGS. 2B-2E, the statistical significance was determined by log-rank test; * p<0.05, ** p<0.01, *** p<0.001, **** p>0.0001; N=5-10/group.

FIGS. 3A-3F display schematics and graphical representation of mice treated with CKM+αPD-1 that develop tumor-specific systemic CTL responses. MC38-bearing mice were treated with PBS (Untx) or CKM+αPD-1 (CKM+αPD-1, sequential) beginning on day 3. Spleen and lymph nodes were harvested 24 hours after the last injection. CD8-selected splenocytes and lymph node cells from untreated and CKM+αPD-1-treated mice were cultured with 4T1 (Off Target) or MC38 cells. Following overnight incubation with 50 U/ml IL-2 (interleukin-2), samples were assayed for IFNγ production (interferon gamma).

FIG. 3A displays a schematic of spots used for quantification of FIG. 3B. FIGS. 3B-3C display the number of spots per 100,000 CD8+ cells. Statistical significance was determined by unpaired t test; * p<0.05; N=3/group. Quantification of IFNγ producing CD8-selected splenocytes (FIG. 3B) and lymph node cells (FIG. 3C). FIG. 3D displays a change in tumor size calculated by subtracting the tumor size on day 15 from the largest tumor size measured for each mouse and shown in correlation to the number of spots counted from the splenocytes and lymph node cells. FIG. 3E displays results of mice inoculated with 500,000 MC38 cells intraperitoneally and treated with sequential regimen consisting of represents two doses of CKM (50 μg rintatolimod and 10,000 IU IFNα) one day apart followed by three doses of αPD-1 (200 μg) one, five, and ten days after the second CKM dose. All treatment was administered intraperitoneally. Controls received intraperitoneal injections of PBS at the same volume as the experimental group. At 90 days (indicated by arrow), surviving mice in the CKM+αPD-1 group were rechallenged with 1,000,000 MC38 cells and monitored for survival. No PBS-treated mice survived to the 90-day timepoint for rechallenge. N=9 at time of rechallenge. FIG. 3F displays results of spleens from mice inoculated with subcutaneous MC38 and regressing tumors following systemic CKM+αPD-1 beginning on day 3 were harvested on after day 90. CD8+ splenocytes were cultured with media alone (Media), unrelated target cells (E0771), or MC38 cells and evaluated for IFNγ production by ELISA. Statistical significance was determined by one-way ANOVA; ** p<0.01, *** p<0.001; N=5/group.

FIG. 4A-4F display schematic and graphical representations of therapeutic synergy between CKM and PD-1 blockade requiring their simultaneous administration or immediate pre-treatment with CKM: Loss of the synergy upon delayed application. Mice were inoculated subcutaneously with MC38 cells. As shown in FIG. 4A, beginning on day 3, mice were treated with CKM in combination with αPD-1 based on the schematics depicted.

Mice experiencing a regression were defined as mice who displayed a tumor that was eliminated by treatment. Non-responders were defined by uncontrolled tumor growth following treatment. FIG. 4B displays standard concomitant schedule as shown in FIG. 2A-2E. FIGS. 4C-4F display a standard sequential schedule as shown in FIG. 2D and modified to include αPD-1 immediately following CKM (FIG. 4C), three days after CKM (FIG. 4E), or five days after CKM (FIG. 4F). N=6-8/group.

FIG. 5A-5D display graphical representations of the anti-tumor effects of CKM in combination with PD-1 blockade require intact immune system and endogenous Batf3-containing cDCs. SCID (FIG. 5A), BATf3 knockout (KO) (FIG. 5B), CCR5 knockout (FIG. 5C), CXCR3 knockout (FIG. 5D) and C57BL/6 wildtype (FIG. 5A-5D) mice were inoculated subcutaneously with MC38 cells and treated with CKM+αPD-1 or PBS control. All treatment was administered intraperitoneally. Statistical significance was determined by log-rank test; ** p<0.01, *** p<0.001, **** p>0.0001; N=5-10/group.

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, process step, and electronic changes may be made without departing from the scope of the disclosure.

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

Although claimed subject matter will be described in terms of certain examples, other examples, including examples that do not provide all of the benefits and features set forth herein, are also within the scope of this disclosure. The steps of the method described in the various examples and examples disclosed herein are sufficient to carry out the methods of the present invention. Thus, in an example, the method consists essentially of a combination of the steps of the methods disclosed herein. In another example, the method consists of such steps.

As used in the specification and the appended claims, the singular forms “a” “and” and “the” include plural referents unless the context clearly dictates otherwise. Ranges may be expressed herein as from “about” one particular value, and/or to “about” another particular value. When such a range is expressed, another example includes from the one particular value and/or to the other particular value. Similarly, when values are expressed as approximations, by the use of the antecedent “about” or “approximately” it will be understood that the particular value forms another example. The term “about” and “approximately” in relation to a numerical value encompass variations of +/−10%, +/−5%, or +/−1%.

Any result obtained by using a described combination as described herein can be compared to a suitable control. In examples, the control is a result obtained using only one of CKM or an anti-PD1 agent. In examples the control comprises or consists of sequentially administering CDM and an anti-PD1 agent, but wherein the anti-PD1 agent is not administered until more than 3, 4, 5, 6, or more days after the CKM. In an example, an improved result can comprise a reduction in tumor volume using the described combination approach, relative to the control, or an inhibition of tumor growth, relative to the control, or sensitization to anti-PD-1 therapy relative to the control, or a synergistic anti-tumor response when using the described combination according to the described administration order and timing, and wherein the control does not result in a synergistic effect

In examples, the anti-PD-1 agent is pembrolizumab, nivolumab, or cemiplimab. In examples, the CKM comprises or consists of a combination of a TLR3 agonist and an interferon (IFN) such as Type 1 IFN-alpha (IFNα). In examples, the TLR3 agonist comprises or consists of poly IC or a poly IC analog, such as rintatolimod which is sold under the tradename Ampligen®.

In examples, the described method uses a therapeutically effective amount of a described combination of described agents. The term “therapeutically effective amount” as used herein refers to an amount of an agent or combination of agents sufficient to achieve, in a single or multiple doses, the intended purpose of treatment. The amount required may vary based on its mode of administration, patient specifics and the like. Appropriate effective amounts can be determined by one of ordinary skill in the art informed by the instant disclosure using routine experimentation. For example, a therapeutically effective amount, e.g., a dose, can be estimated initially either in cell culture assays or in animal models. An animal model can also be used to determine a suitable concentration range, and route of administration. Such information can then be used to determine useful doses and routes for administration in humans, or to non-human animals. A precise dosage can be selected by in view of the patient to be treated. Dosage and administration can be adjusted to provide sufficient levels of the combination of the described agents to achieve a desired effect.

Additional factors which may be taken into account include the type or stage of cancer, the age, weight and gender of the patient, reaction sensitivities, and tolerance/response to therapy. In examples, a therapeutically effective amount is an amount that reduces one or more signs or symptoms of cancer, and/or reduces the severity of the cancer. A therapeutically effective amount may also inhibit or prevent the cancer relapse. In examples, a described combination administered according to the described sequence and time periods may result in using less of the anti-PD-1 agent than would be administered without the CKM combination. In examples, a described combination administered according to the described sequence and time periods may result in a greater than additive effect that if the CKM and the anti-PD-1 agent were administered sequentially but not in combination over a described time period. In examples, the described combination may be administered using the same, or different routes, including but not necessarily limited to intravenous administration.

In examples this disclosure demonstrates that use of both local or a systemic CKM combination, but not their individual components effectively sensitize PD-1-resistant established MC38 or CT26 tumors to PD-1 blockade. CKM+αPD-1 combination promoted local influx of Batf3-positive cDC1s and CTLs, inhibited tumor growth and prolonged survival, inducing cures in 20-100% of the treated animals, depending on the tumor model, time of the onset of treatment (early-or late-stage disease), and the route of delivery (local or systemic). CKM+αPD-1-treated mice developed local and systemic tumor-specific CTL responses and resistance to tumor re-challenge.

In examples, and as described above, the described effectiveness of CKM comprises its administration at the time of, or directly before, PD1 blockade and is strongly reduced by 24-hour delay between CKM and PD1 blockade. Thus, in a representative examples, CKM is administered to a patient first, following the administration of the anti-PD-1 agent. The CKM may be administered on the same day as the anti-PD-1 agent (provided CKM is administered first), or not more than one, two or three days after the CKM, and preferably within one day. In examples, the doses of CKM and the anti-PD-1 agent are both administered within a two-day period. In examples, the CKM is administered as a first dose, which may be followed by at least one additional CKM dose, after which the anti-PD-1 agent inhibitor is administered within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours, including all times and ranges of time points there between, of the CKM. In examples, an individual may be treated with a described consecutive treatment for a period, followed by a period wherein neither CKM or the anti-PD-1 agent is administered, or only one of CKM or the anti-PD-1 is administered. In an example, such period may be 1-14 days. In an example, CKM is administered for three cycles, followed by CKM and an anti-PD-1 agent administered according to the described time period. After the initial one or several CKM administration, administration of the anti-PD-1 agent is performed according to a described time period, and may be repeated one or more times before the CKM is administered again.

As demonstrated by the description and figures of this disclosure, the CKM-driven intratumoral CTL accumulation and antitumor effects were abrogated in animals lacking Batf3-positive cDC1s, CD8+ T cells, or CXCR3 and CCR5, the CTL-expressed receptors for CXCL9/CXCL10 and CCL5. The demonstration that both local and systemic CKM can promote the therapeutic effectiveness of PD1 blockade in nominally PD1-resistant “cold tumors”, indicate that intratumoral CTL accumulation, rather than lack of sufficient antigenic targets is the dominant factor limiting the therapeutic effectiveness of ICI in mouse tumors. The unexpected observation that the effectiveness of the combination treatment requires close timing of the CKM component and the ICI component that supports the described improved treatment modalities including CKM and ICI and potentially, other forms of immunotherapy.

This disclosure demonstrates that the PD-1 blockade administered systemically at the same time or directly following CKM administration shows strongly enhanced antitumor effects. The present demonstration that tumor-bearing mice respond favorably to systemic CKM+αPD-1 treatment supports the use of systemic CKM widely to sensitizing tumors to immune-based therapies, even if tumors cannot be directly accessed. The demonstration that the effects of CKM require its administration at the same time or directly before αPD-1 treatment provides for a preferred implementation of the described approach.

### Example 1

The following materials and methods were used to produce the results described in this disclosure.

### Mice

Female 6-8 week old C57BL/6J (JAX stock #000664), BALB/cJ (JAX stock #000651), B6.129P2-Ccr5^{tm1Kuz}/J (CCR5 KO; JAX stock #005427), and B6.129P2-Cxcr3^{tm1Dgen}/J(CXCR3 KO; JAX stock #005796) mice were purchased from The Jackson Laboratory and experiments began within 2 weeks of arrival. Female 6-8 week old C. B-Igh-1bIcrTac-Prkdc^{scid}/Ros (SCID) and NOD. Cg-Prkdc^{scid}I12rg^{tm1Wjl}/SzJ (NSG) mice were bred through the Roswell Park Comprehensive Cancer Center Comparative Oncology Shared Resource. All mice were maintained in specific pathogen-free conditions in a HEPA-filtered ventilated rack system at the Roswell Park Comprehensive Cancer Center Comparative Oncology Shared Resource. Mice were housed 5 or less per cage with Enrich-o'Cobs ¼ (The Andersons) enrichment. Sterile pellet Teklad Global 2018SC Rodent Diet (Inotiv) and water were provided ad libitum. Mice were maintained on a 12-hour light/dark cycle at 24 °C±2° (~70 °F) and 50% average humidity (range 30-70%). All in vivo experiments were performed in accordance with institutional guidelines under protocols approved by the Institutional Animal Care and Use Committee at Roswell Park Comprehensive Cancer Center.

### Cell Lines

MC38 (Kerafast) and CT26 (American Type Culture Collection) were obtained directly from the manufacturer and stored frozen in aliquots after <5 passages. Cells were cultured in RPMI-1640 with GlutaMAX™(Gibco) supplemented with 10% fetal bovine serum and other additives according to manufacturer recommendations. Prior to inoculation, cells were harvested at ~90% confluency at <10 passages from the original stock. For in vivo work, freshly harvested tumor cells were suspended in PBS and injected subcutaneously into the left flank or intraperitoneally. Subcutaneous injections of both CT26 and MC38 cells were administered at 500,000 cells per 50 μl PBS for initial inoculation. Intraperitoneal injections of MC38 were given at 500,000 cells per 200 μl PBS. For rechallenge, mice surviving following treatment of intraperitoneal tumors were inoculated with 1,000,000 MC38 cells per 50 μl PBS subcutaneously.

### Treatment Strategy

Following subcutaneous tumor implantation, mice were monitored daily to identify palpable tumors at which time they were randomized into treatment groups. To model early stage CRC, treatments were administered beginning on Day 3. To model late stage CRC, treatments were administered to mice bearing palpable tumors at ~10-14 days following inoculation (tumor volume ~20-100 mm³). Rintatolimod (provided by AIM ImmunoTech Inc.) was administered in 50 μg doses, IFNα (Miltenyi Biotec) was administered in 10,000 IU doses, and αPD-1 (clone RMP1-14; Leinco Technologies, Inc.) was administered in 200 μg doses. For concomitant administration, three cycles consisting of two doses of CKM (50 μg rintatolimod and 10,000 IU IFNα) one day apart were given, followed by 200μg αPD-1 the next day. For sequential treatment, two doses of CKM (50 μg rintatolimod and 10,000 IU IFNα) were given one day apart followed by three doses of αPD-1 (200 μg) one, five, and ten days after the second CKM dose. When administering CKM or αPD-1 alone, doses were administered on the corresponding days these treatments were given in the combinatorial schedule of CKM+αPD-1. For mice receiving PBS control, 200 μl sterile PBS was administered on all days and treatment was administered to comparative groups of mice. When rintatolimod and IFNα were given together, they were administered in one intraperitoneal injection. All treatment was administered intraperitoneally in a volume of 200 μl. Tumors were measured with digital calipers and tumor volume was calculated in mm³ using the formula (length*width*width)/2. Following the initiation of treatment, tumors were measured at least twice a week until size reached 1000 mm³ at which point measurements were taken daily. The endpoints for survival studies included when tumor volume reached >1500 mm³, mice became lethargic, or ascites developed prohibiting movement. Endpoints were noted when subcutaneous tumors reached 1500 mm³ or the longest diameter reached 2 cm for subcutaneous tumors. For intraperitoneal tumors, endpoints were noted when the diameter or shape of the abdomen has clearly changed (abdominal distension). All mice were humanely euthanized at endpoints in accordance with the Institutional Animal Care and Use Committee at Roswell Park Comprehensive Cancer Center.

### RT-qPCR

The expression of mRNA was analyzed using TaqMan-based RT-qPCR. For in vitro analysis, MC38 cells were cultured and subsequently treated in DMEM media enriched with 10% FBS, HEPES, NEAA, sodium pyruvate, l-glutamine, and antibiotics (penicillin streptomycin and gentamycin sulfate). For ex vivo analysis of splenocytes, spleens were excised from mice following treatment. Spleens were digested and red-blood cell lysed before incubation and treatment in AIM-V supplemented with antibiotics (penicillin streptomycin). Splenocytes were treated with 50 μg rintatolimod, 10,000 IU IFNα, or a combination of both. For ex vivo total TME analysis, tumors were excised from mice following treatment. All samples were processed in RLT buffer (Qiagen) and homogenizing tubes were used for digestion of tumors. Supernatants were collected and total RNA was extracted using RNeasy kit (Qiagen). For RNA, 250 ng per cDNA synthesis reaction was used to generate cDNA with a qScript cDNA Synthesis kit (QuantaBio) and the resulting cDNA was diluted 5× in DNase/RNase-free water for mRNA expression analysis. For ex vivo analysis of TILs and tumor cells, CD8+cells were prior to processing in RLT buffer. TaqMan primers (Thermo Fisher Scientific) included: CD8α(Mm01182107_g1), CCL5(Mm01302427_m1), CCL22 (Mm00436439_m1), CCR5(Mm01963251_s1), CXCL9(Mm00434946_m1), CXCL10 (Mm99999072_m1), CXCR3 (Mm00438259_m1), FoxP3(Mm00475162_m1), GZMB (Mm00442837_m1), and IFNγ (Mm01168134_m1).

### ELiSPOT

Splenic MC38-Ag specific^{CD8+} T cells were quantified via IFNγ production. Spleens were excised and single cell suspension was generated using a screen before ACK-lysis. CD8-selected splenocytes were incubated overnight with low-dose IL-2. MC38-Ag specificity was determined using a negative control cell line (4T1). Spots were counted to calculate total number of splenic Ag-specific^{CD8+} T cells.

### ELISA

To measure IFNγ production of splenic^{CD8+} T cells, spleens were excised, and single cell suspension was generated using a screen before ACK lysis. ^{CD8+}splenocytes were incubated overnight with low-dose IL-2. Effector cells (^{CD8+}splenocytes) and target cells (media, E0771, MC38) were incubated for 48 hours before ELISA. IFNγ production was quantified using R&D Systems Mouse IFN-γ ELISA kit (DY485).

### Statistics

One way ANOVA with Tukey's post-test for multiple comparisons was used to compare readouts from four groups and unpaired t tests were used to compare readouts between two groups. All p values less than 0.05 are reported and indicated on the figures. Kaplan-Meier survival plots and log-rank tests were used to display and analyze the time from the beginning of treatment to death or the date of sacrifice. All tests were two-sided. All analyses were performed using GraphPad Prism version 10.

The following is a description of results obtained using the materials and methods described above.

### Example 2

Combinatorial CKM selectively induces CTL-attracting chemokines in mouse TME in vitro and enhances intratumoral CTL-attractants and CTL markers in vivo upon its systemic application in tumor-bearing mice.

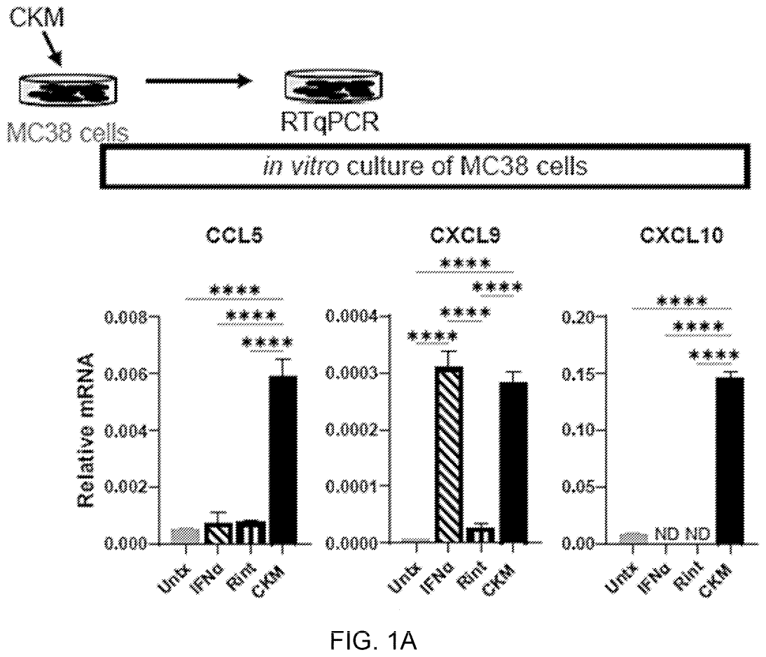

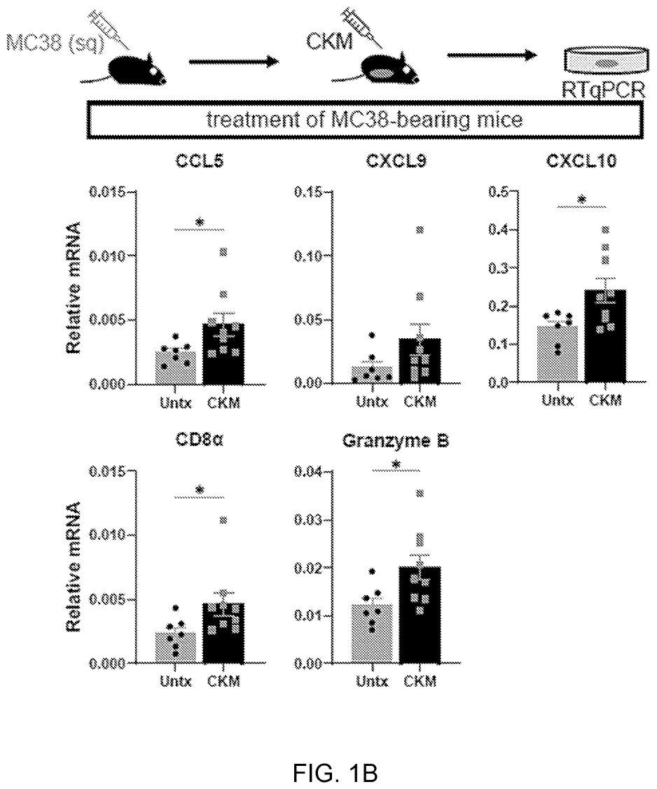

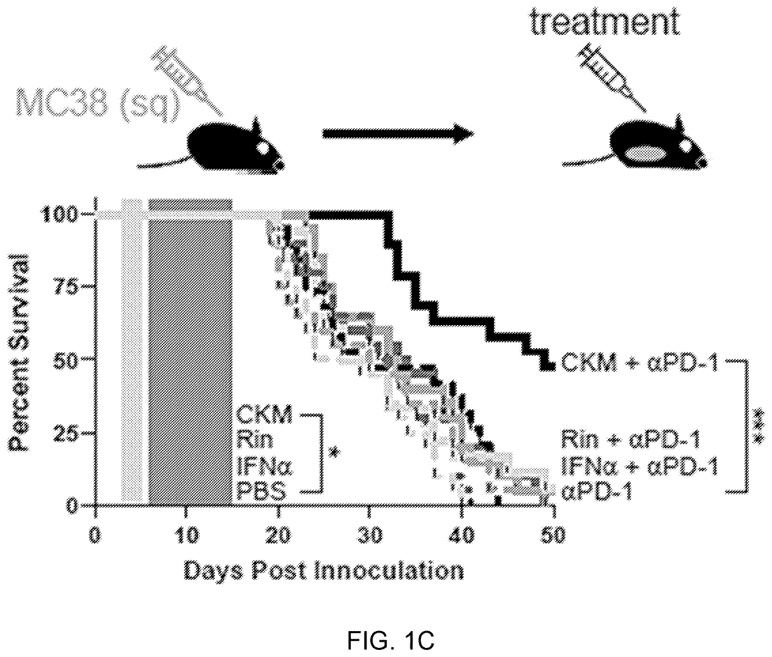

To determine the susceptibility of mouse CRC cells to CKM, we examined the impact of treatment on MC38 cells in culture. Cells were treated with IFNα, rintatolimod, or a combination of the two (CKM) to evaluate their synergy (FIG. 1A). Expression of chemokines known to attract CTLs, including CCL5, CXCL9, and CXCL10 were significantly upregulated following treatment with CKM (FIG. 1A). IFNα alone upregulated expression of CXCL9 to a similar extent as the combinatorial CKM but the induction of CCL5 and CXCL10 was strictly dependent on the double activation by both CKM components (FIG. 1A), indicating that optimal TME modulation requires a combinatorial approach.

To test if similar changes can be also induced in mouse tumors in vivo, mice with established subcutaneous MC38 tumors were treated with one dose of intraperitoneal CKM. Tumors were harvested three days after treatment and mRNA expression was evaluated using RT-qPCR (FIG. 1B). As predicted by our ex vivo data, systemic CKM enhanced local intratumoral expression of CTL-attracting chemokines CCL5 and CXCL10 with additional trend towards increased CXCL9 expression (FIG. 1B). In addition, systemic CKM treatment promoted intratumoral increases of CTL markers: CD8α and granzyme B (FIG. 1B).

Our previous work has shown that a combination approach, including both TLR-3 stimulation (i.e., poly: IC or rintatolimod) and type 1 IFN (i.e., IFNα), reprograms the TME of various human and mouse tumors^{34-36, 40, 41}. We tested this concept in vivo and found slowed tumor growth (FIG. 1C) and longer survival (FIG. 1C) when applying both rintatolimod and IFNα. While the in vivo effects of CKM on mouse CRC were statistically significant, the impact was unimpressive. For synergistic action of both IFNα and rintatolimod in the induction of CTL attractants (FIG. 1A), the therapeutic effects required combined application of rintatolimod and IFNα, in addition to PD1 blockade (FIG. 1C).

### Example 3

Local and systemic administration of CKM converts nominally PD-1-resistant tumors into PD1-responsive ones: Synergy between IFNα and rintatolimod.

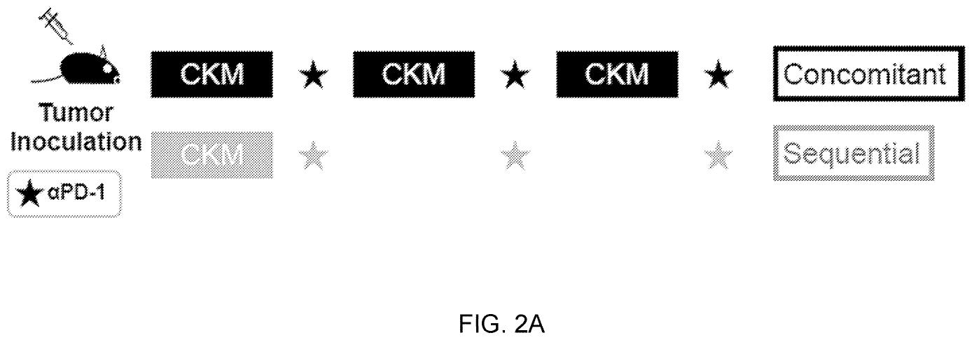

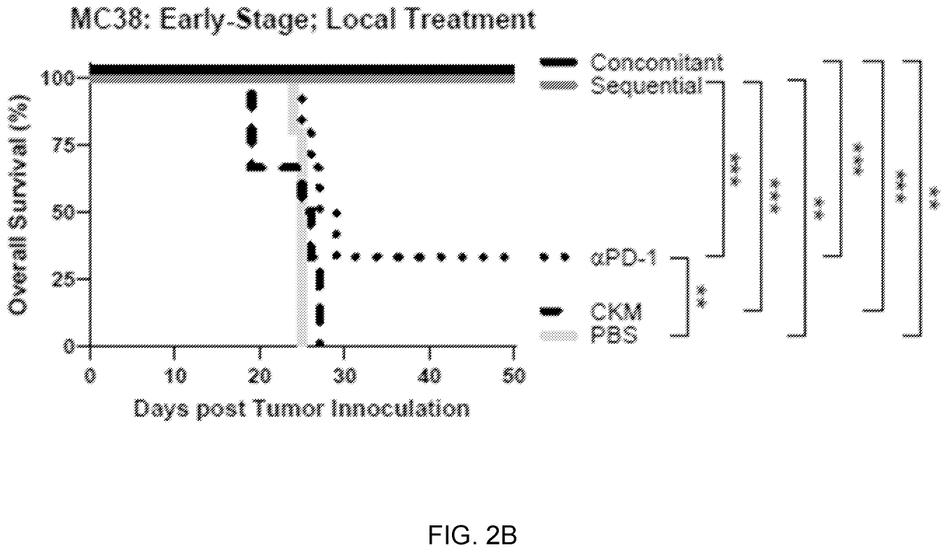

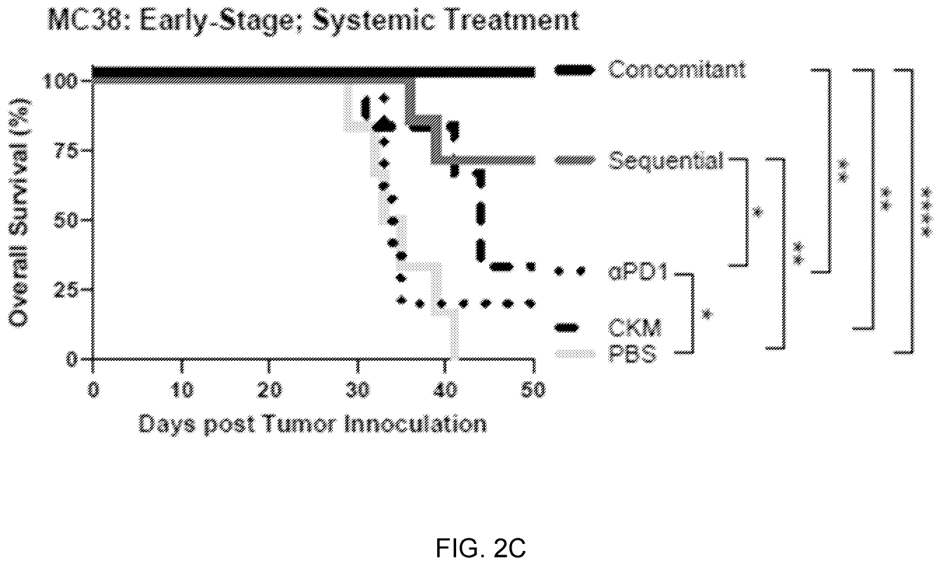

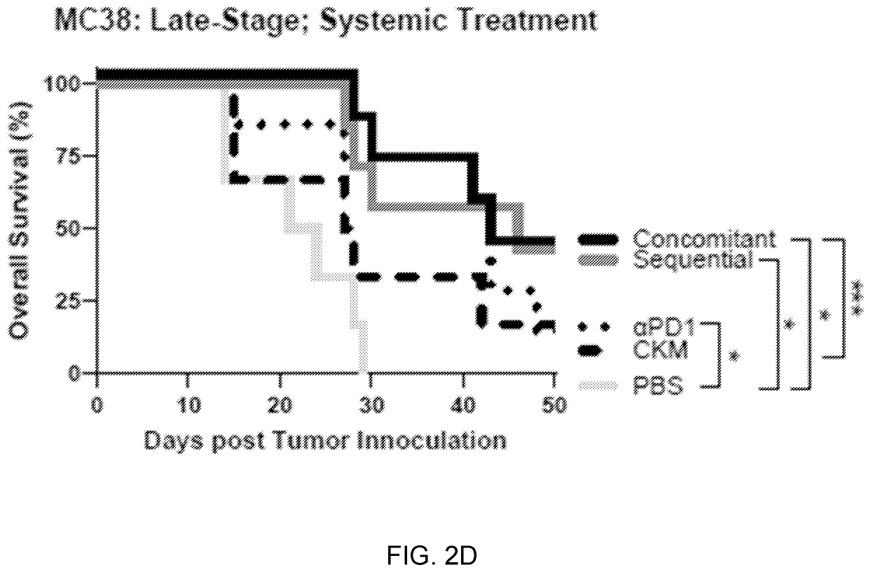

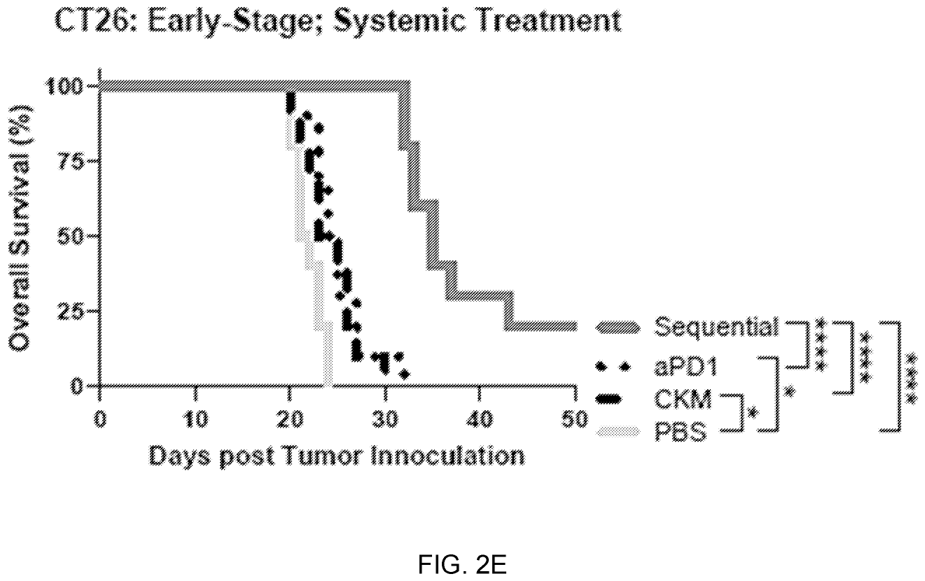

To test the efficacy of adding PD-1 blockade to CKM for treatment of mouse CRC, we compared two treatment schedules for combination of CKM and αPD-1. As shown in FIG. 2A, we developed a concomitant schedule where CKM was administered directly prior to αPD-1 for three cycles and a sequential schedule where CKM was administered first followed by three subsequent αPD-1 injections. In addition to evaluating the schedule for administrating CKM and αPD-1 in combination approaches, we also tested the effectiveness of local (FIG. 2B) and systemic (FIG. 2C-E) treatments by inoculating tumors via intraperitoneal or subcutaneous routes, respectively. Local treatment of early-stage MC38 tumors, where treatment is started three days after inoculation, resulted in complete abrogation of all mice treated by either concomitant or sequential schedules (FIG. 2B). When using the systemic model defined by early-stage subcutaneous tumors receiving intraperitoneal treatment, both concomitant and sequential schedules demonstrated statistically better efficacy than monotherapies (FIG. 2C). In this setting, concomitant drug administration did result in the optimal tumor control in 100% of tumor-bearing mice, while the sequential administration where the αPD-1 component was administered directly after the CKM resulted in only 70% effectiveness (FIG. 2C). We next explored the effects of CKM+αPD-1 on more established, late-stage MC38 tumors. When treating established tumors, both concomitant and sequential treatment cured approximately half of treated mice within 50 days of tumor inoculation (FIG. 2D).

Finally, to determine if the efficacy of CKM+αPD-1 can be extended from the relatively immunogenic MC38 cell line, most commonly used to optimize immune treatment approaches⁴⁸⁻⁵¹ (which showed partial responsiveness to PD1 alone), to fully PD1-resistant CRC, we tested the efficacy of the CKM+αPD-1 regimen on established CT26 CRC. We found that CKM+αPD-1 still uniformly delayed the progression and induced cures in 25% of the animals with CT26 tumors, which were fully resistant to PD1 blockade alone (FIG. 2E).

### Example 4

CKM+αPD-1 treatment activates lymphoid tissues and induces systemic tumor-specific CTL responses.

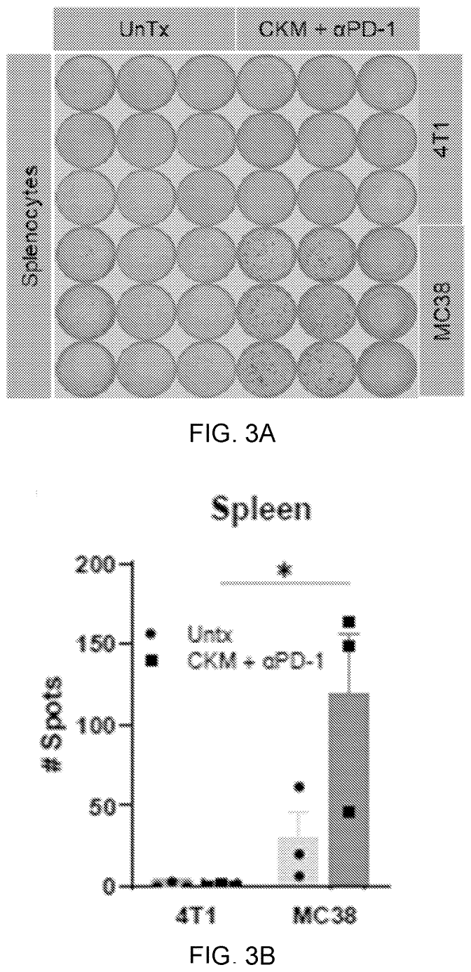

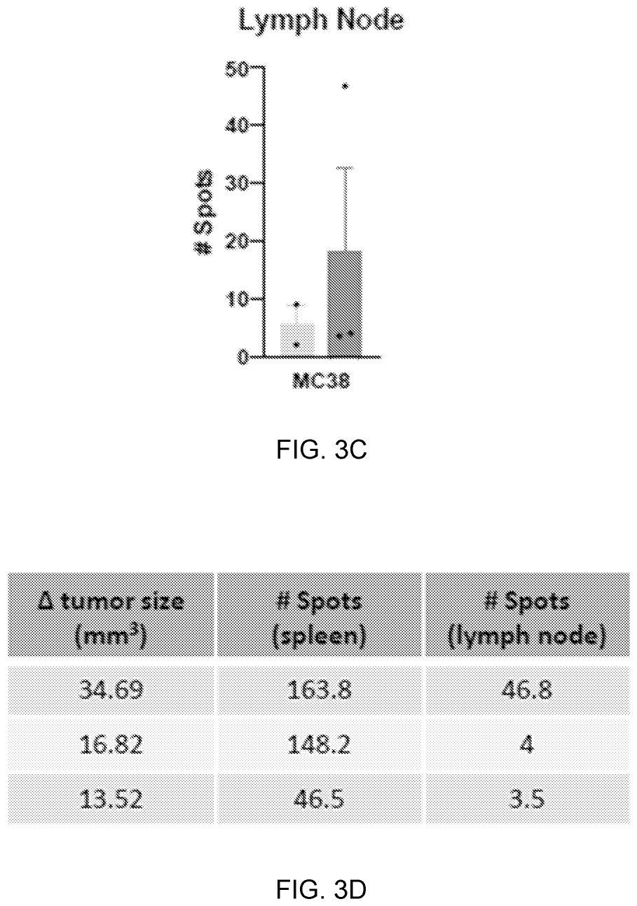

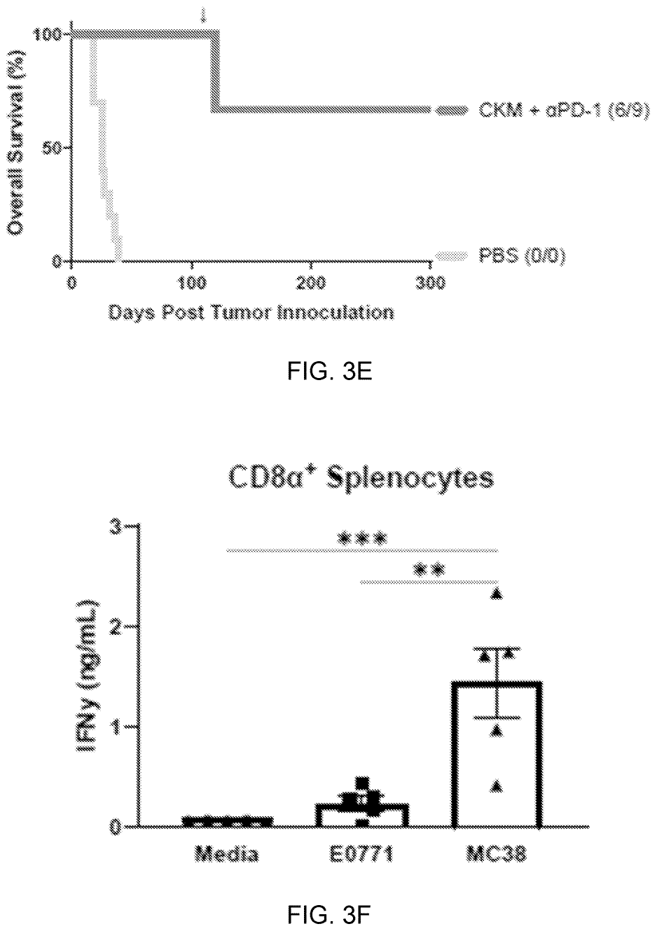

We next evaluated production of IFNγ from splenocytes and lymph nodes of mice treated with CKM+αPD-1 therapy. Organs were harvested 24 hours following the final treatment at which time treated mice were demonstrating tumor regression. CD8-selected splenocytes from treated and untreated MC38-bearing mice were cultured ex vivo with off target (4T1) cells, during with no IFNγ production was observed (FIGS. 3A-B). However, when cultured in the presence of MC38 cells, CD8-selected splenocytes (FIGS. 3A-B) and lymph node cells (FIG. 3C) from two of three treated mice demonstrated a notable increase in IFNγ production. Importantly, the percentage (~67%) of responding mice corresponds with our observations of long-term responses from treating early-stage MC38 tumor with sequential administration of CKM+αPD-1 (FIG. 2B). The mice which experienced the largest tumor regressions also had the highest production of IFNγ (FIG. 3D).

Unexpectedly, when rechallenged with double the number of the same MC38tumor cell line, 66.7% of surviving mice rejected the original tumors (FIG. 3E), suggesting that, in addition to local attraction of CTLs, CKM may also promote the induction of tumor-specific systemic immunity. In addition, CD8+ splenocytes from long-term (over 90 day) survivors who experienced tumor regression following CKM+αPD-1 treatment exhibited MC38-antigen specific IFNγ production (FIG. 3F). Taken together, these findings demonstrate that CKM+αPD-1 therapy can induce favorable immune changes which occur quickly after treatment but also result in long-lasting immunity.

### Example 5

Transient character of the CKM-induced window of susceptibility to PD-1 blockade results in the need for concomitant or back-to-back treatment.

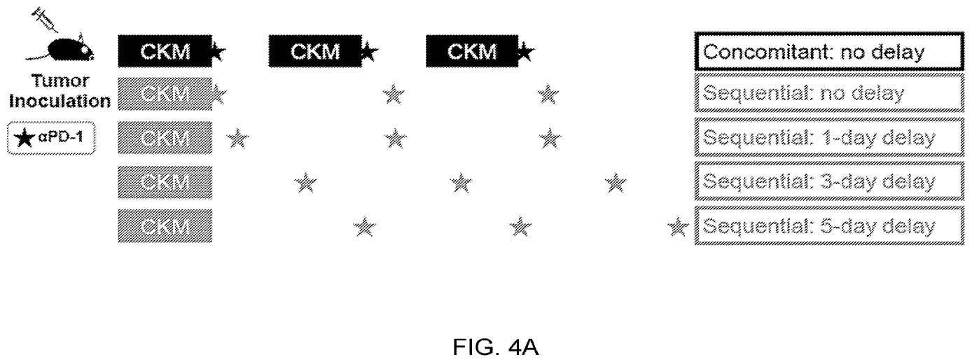

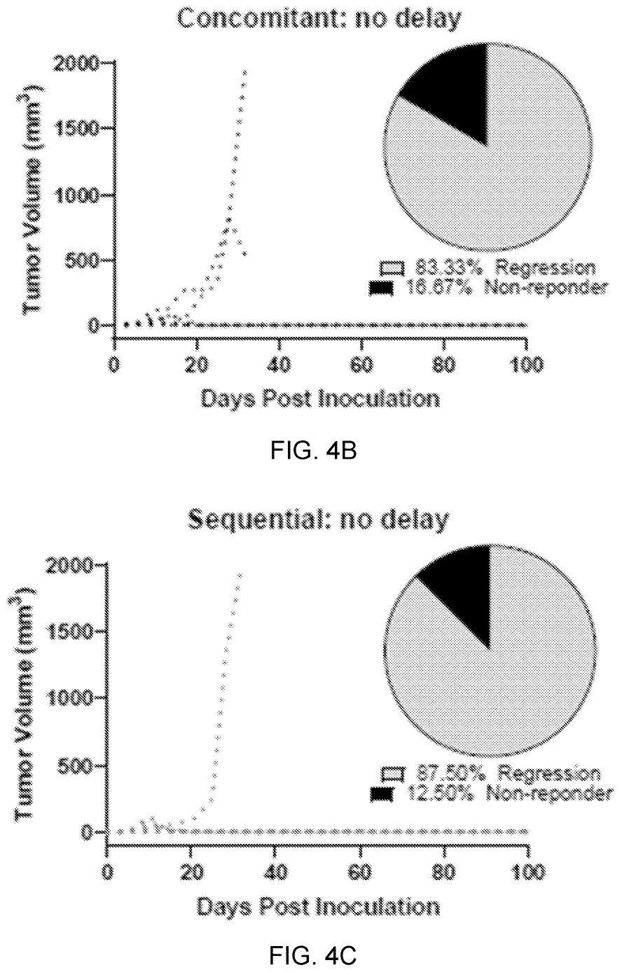

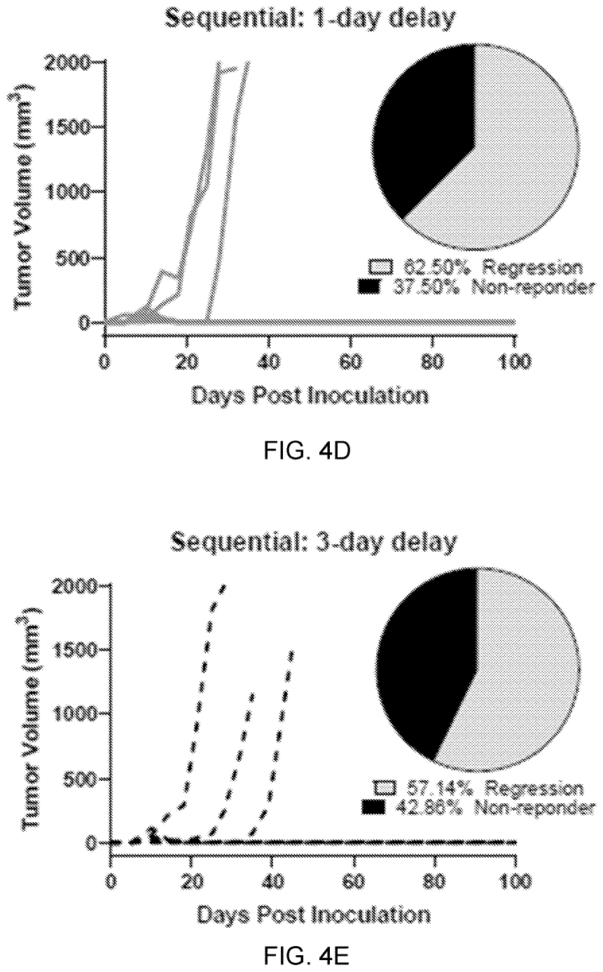

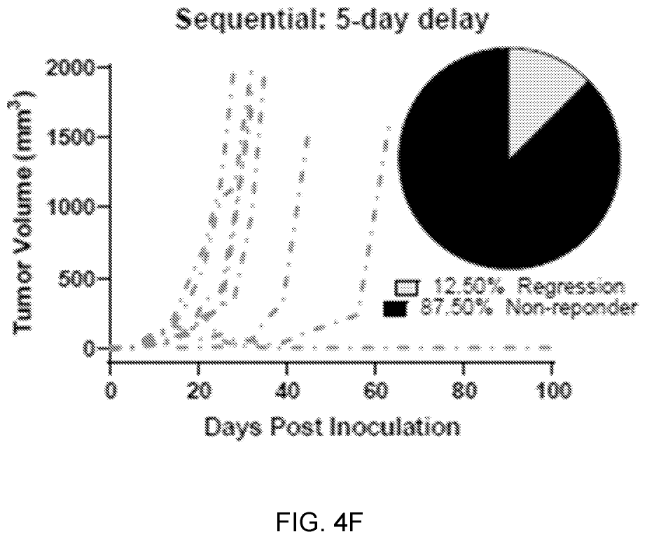

To test the duration of the CKM-induced window of sensitization of tumors to PD1 blockade, we compared the effectiveness of αPD-1 administered at the same time or at different timepoints after CKM completion of treatment (FIG. 4A). Unexpectedly, we found CKM-induced responsiveness to PD-1 blockade occurred almost immediately after treatment (FIGS. 4B-C). We saw more responding mice when αPD-1 was administered right after CKM (FIG. 4C) compared to when αPD-1 was administered one day after CKM (FIG. 4D). Similarly, using sequential administration, fewer mice experienced tumor regression when αPD-1 was delayed three or five days after CKM administration (FIGS. 4E-F). These data demonstrate that αPD-1 could effectively be administered on the same day as CKM, a strategy likely to ease clinical translation and compliance when designing subsequent clinical trials.

### Example 6

Anti-tumor effects of CKM+αPD-1 combination require intact immune system, including endogenous Batf3-containing cDCs, CCR5, and CXCR3.

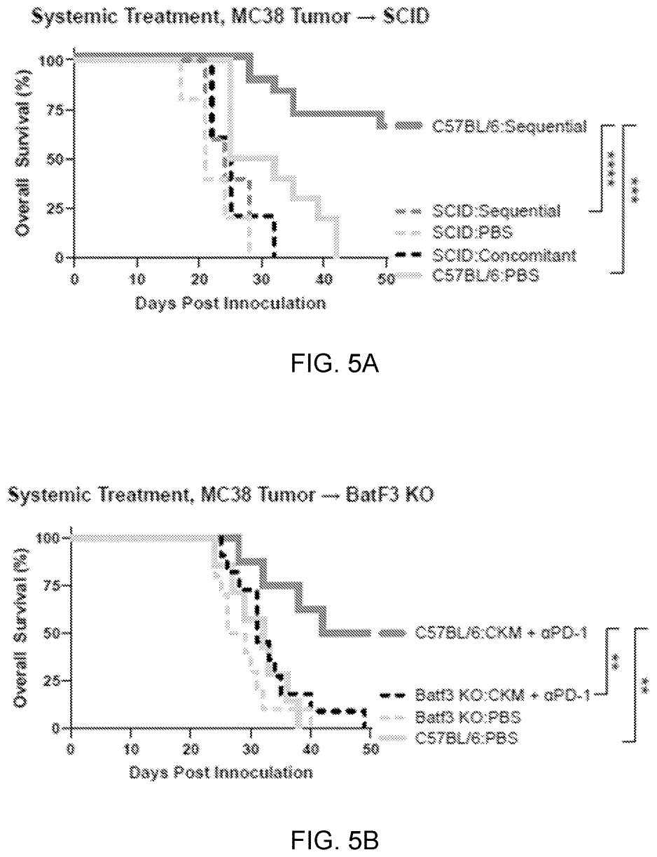

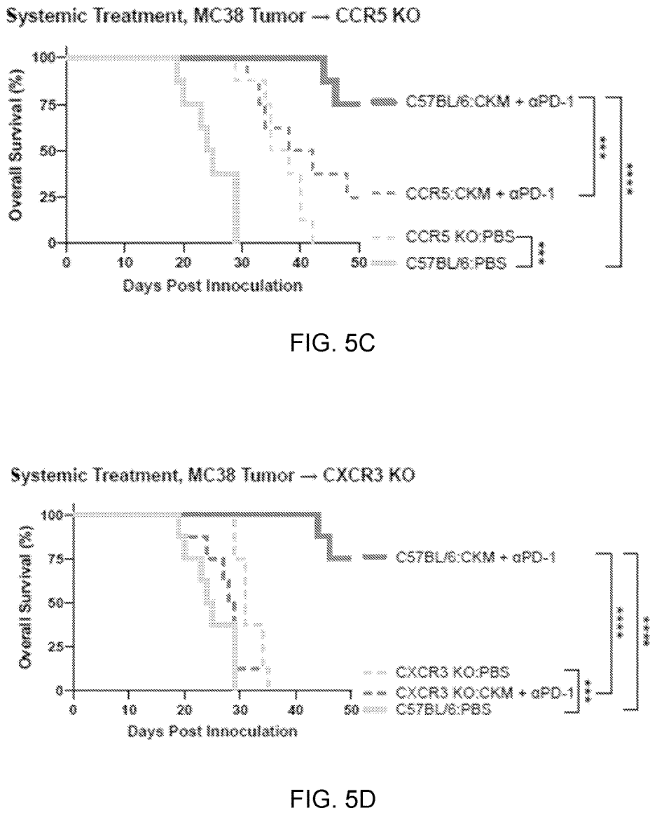

T cells and DCs play a pivotal role in regulating anti-tumor immune response⁵². We found no effect of CKM+αPD-1 treatment to MC38-bearing immunodeficient SCID⁵³ (FIG. 5A) mice, indicating a therapeutic dependence on immune cell subsets, including T cells or NK (natural killer) cells. We found endogenous CD8α conventional DCs (cDCs) were required for the therapeutic efficacy of CKM+αPD-1 based on the lack of anti-tumor responsiveness in Batf3^{-/-} mice⁵⁵ (FIG. 5B). Because cross-priming of CD8+ T cells requires Batf3^{56,57}, these findings suggest that antigen cross-presentation by cDCs to CTLs represents a vital component of CKM+αPD-1 efficacy against mouse CRC.

To further elucidate the impact of CKM+αPD-1 on chemokine modulation, we investigated the role of two important chemokine receptors; CCR5, a ligand for CTL-attracting chemokine CCL2⁵⁸, and CXCR3, a ligand for CTL-attracting chemokines CXCL9/10⁵⁹. We found that mice lacking CCR5 do not garner as robust of a response to CKM+αPD-1 therapy as WT C57BL/6 mice, although, there is some efficacy of the treatment (FIG. 5C). However, mice lacking CXCR3, exhibit no response to CKM+αPD-1 therapy (FIG. 5D). Taken together, these findings indicate a minor role for the CCR5/CCL5 interaction and a more prominent role of the CXCR3/CXCL9/10 axis in the effects of this treatment.

It will be recognized from the foregoing examples and description that the present disclosure demonstrates that CKM, a combination therapy consisting of a TLR3 agonist (rintatolimod) and Type 1 IFN (IFNα) can induce CTL-attracting chemokines in murine CRC tumors. This treatment strategy is also associated with favorable immune changes in the TME, including an induction of CD8α, Granzyme B, and TNFα message. When PD-1 blockade is added to CKM, we show substantial tumor control and induction of anti-tumor immune responses. Both local and systemic administration of this combinatorial treatment regimen results in durable regression of MC38 tumors. Even late-stage tumors show a modest response (~50%) to the CKM+αPD-1 therapeutic regimen. In contrast to our original expectations resulting from our previous work³¹⁻⁴², which led to the design of our recent clinical trials in triple negative breast cancer patients (NCT03599453)⁴² and colorectal cancer patients (NCT04119830) where PD1 blockade was started within days to weeks after completion of CKM treatment the currently provided and unexpected data show that the antitumor effectiveness of the combined CKM and αPD-1 therapy is dependent on the timing of its components and works best when αPD-1 was administered at the time of immediately after CKM (FIGS. 4A-4F).

The data also indicate that mice experiencing tumor regression following CKM+αPD-1 treatment can generate durable immunity. Treated mice cured of their tumor exhibit long-lasting capacity to upregulate immune factors, including CD8α, Granzyme B, IFNγ, and TNFα. Further, splenocytes from treated and cured mice still upregulate CTL-attracting chemokines (CCL5, CXCL9, CXCL10) in response to CKM more than 90 days after tumor inoculation.

The disclosure demonstrates a requirement of two chemokine receptors for effective CKM+αPD-1 treatment. The described treatment combination shows a partial dependence on CCR5 (FIG. 5C), the ligand for CTL-attracting chemokine CCL5⁵⁸. CCR5 has been shown to be a necessary signaling component for macrophage-driven expression of inflammation in response to double-stranded RNA⁶¹, and since our treatment regimen contains rintatolimod, a double-stranded RNA which activates TLR3⁴⁴, it is not surprising that CCR5 deficiency impedes the effects of CKM+αPD-1 therapy. While CCL5/CCR5 signaling does facilitate recruitment of CTLs to the TME, it has also been implicated in the recruitment of Tregs⁶² and thus, its depletion could be, in part, combating immunosuppressive effects.

The disclosure shows a role for chemokine receptor CXCR3 (FIG. 5D), which serves as a ligand for T cell attracting chemokines CXCL9/10/11 and is prominently expressed on activated T and NK cells⁵⁹. While CXCR3's absence perturbs T cell trafficking into the TME, some T cells are able to infiltrate the tumor in mouse models and frequencies of T cells in the spleen and lymph node remain consistent with that in wild type tumor-bearing mice⁶⁷. Thus, the complete abrogation of CKM+αPD-1 efficacy in CXCR3 KO mice suggests a broader impact of CXCR3 in this model. CXCR3 has been shown to augment the ability of tissue-localized ^{CD8+} T cells to find abnormal cells and enact effector functions in viral models⁶⁸ and to facilitate autoimmune reactions in murine graft-versus-host disease models^{69.}

Our initial expectation of the combined CKM and αPD-1 therapy was that CKM-induced susceptibility to PD-1 blockade would occur optimally with a delay between CKM and αPD-1 administration, allowing time for T cell priming. However, our data suggests that the therapy regimen worked best when αPD-1 was administered immediately after CKM (FIGS. 4A-4F).

The disclosure demonstrates two efficacious schedules for drug delivery, a benefit that could be capitalized on during clinical trial design and implementation. This provides options to improve feasibility and compliance for patients receiving these therapies in the clinical setting. Treatment administration following the described sequential approach would limit the number of visits a patient would need to make to a clinic for treatment. Further, the data showing that CKM sensitizes colorectal tumors to PD-1 blockade quickly, enables treating patients with CKM and αPD-1 in the same visit for colorectal cancers, as well as other cancers that are initially resistant to anti-PD-1 therapy.

Although the present disclosure has been described with respect to one or more particular embodiments and/or examples, it will be understood that other embodiments and/or examples of the present disclosure may be made without departing from the scope of the present disclosure.

References—this reference listing is not an indication that any reference is material to patentability:
1. Lee, S. M., et al. First-line atezolizumab monotherapy versus single-agent chemotherapy in patients with non-small-cell lung cancer ineligible for treatment with a platinum-containing regimen (IPSOS): a phase 3, global, multicentre, open-label, randomised controlled study. Lancet 402, 451-463 (2023).
2. Cortes, J., et al. Pembrolizumab plus Chemotherapy in Advanced Triple-Negative Breast Cancer. N Engl J Med 387, 217-226 (2022).
3. Seiwert, T. Y., et al. Safety and clinical activity of pembrolizumab for treatment of recurrent or metastatic squamous cell carcinoma of the head and neck (KEYNOTE-012): an open-label, multicentre, phase 1b trial. Lancet Oncol 17, 956-965 (2016).
4. Le, D. T., et al. Phase II Open-Label Study of Pembrolizumab in Treatment-Refractory, Microsatellite Instability-High/Mismatch Repair-Deficient Metastatic Colorectal Cancer: KEYNOTE-164. J Clin Oncol 38, 11-19 (2020).
5. Larkin, J., et al. Overall Survival in Patients With Advanced Melanoma Who Received Nivolumab Versus Investigator's Choice Chemotherapy in CheckMate 037: A Randomized, Controlled, Open-Label Phase III Trial. J Clin Oncol 36, 383-390 (2018).
6. Sharma, P., Hu-Lieskovan, S., Wargo, J. A. & Ribas, A. Primary, Adaptive, and Acquired Resistance to Cancer Immunotherapy. Cell 168, 707-723 (2017).
7. Haslam, A. & Prasad, V. Estimation of the Percentage of US Patients With Cancer Who Are Eligible for and Respond to Checkpoint Inhibitor Immunotherapy Drugs. AMA Netw Open 2, e192535 (2019).
8. Rizvi, N. A., et al. Cancer immunology. Mutational landscape determines sensitivity to PD-1 blockade in non-small cell lung cancer. Science 348, 124-128 (2015).
9. Goodman, A. M., et al. Tumor Mutational Burden as an Independent Predictor of Response to Immunotherapy in Diverse Cancers. Mol Cancer Ther 16, 2598-2608 (2017).
10. Yarchoan, M., Hopkins, A. & Jaffee, E. M. Tumor Mutational Burden and Response Rate to PD-1 Inhibition. N Engl J Med 377, 2500-2501 (2017).
11. Brahmer, J. R., et al. Safety and activity of anti-PD-L1 antibody in patients with advanced cancer. N Engl J Med 366, 2455-2465 (2012).
12. Topalian, S. L., et al. Safety, activity, and immune correlates of anti-PD-1 antibody in cancer. N Engl J Med 366, 2443-2454 (2012).
13. Hamid, O., et al. Safety and tumor responses with lambrolizumab (anti-PD-1) in melanoma. N Engl J Med 369, 134-144 (2013).
14. Herbst, R. S., et al. Predictive correlates of response to the anti-PD-L1 antibody MPDL3280A in cancer patients. Nature 515, 563-567 (2014).
15. Taube, J. M., et al. Association of PD-1, PD-1 ligands, and other features of the tumor immune microenvironment with response to anti-PD-1 therapy. Clin Cancer Res 20, 5064-5074 (2014).
16. Tumeh, P. C., et al. PD-1 blockade induces responses by inhibiting adaptive immune resistance. Nature 515, 568-571 (2014).
17. Huang, A. C., et al. T-cell invigoration to tumour burden ratio associated with anti-PD-1 response. Nature 545, 60-65 (2017).
18. Hirano, F., et al. Blockade of B7-H1 and PD-1 by monoclonal antibodies potentiates cancer therapeutic immunity. Cancer Res 65, 1089-1096 (2005).
19. Davis, A. A. & Patel, V. G. The role of PD-L1 expression as a predictive biomarker: an analysis of all US Food and Drug Administration (FDA) approvals of immune checkpoint inhibitors. J Immunother Cancer 7, 278 (2019).
20. Yi, M., et al. Biomarkers for predicting efficacy of PD-1/PD-L1 inhibitors. Mol Cancer 17, 129 (2018).
21. Fox, B. A., et al. Defining the critical hurdles in cancer immunotherapy. J Transl Med 9, 214 (2011).
22. Maleki Vareki, S. High and low mutational burden tumors versus immunologically hot and cold tumors and response to immune checkpoint inhibitors. J Immunother Cancer 6, 157 (2018).
23. Samstein, R. M., et al. Tumor mutational load predicts survival after immunotherapy across multiple cancer types. Nat Genet 51, 202-206 (2019).
24. Jardim, D. L., Goodman, A., de Melo Gagliato, D. & Kurzrock, R. The Challenges of Tumor Mutational Burden as an Immunotherapy Biomarker. Cancer Cell 39, 154-173 (2021).
25. McGrail, D. J., et al. High tumor mutation burden fails to predict immune checkpoint blockade response across all cancer types. Ann Oncol 32, 661-672 (2021).

26. Ascierto, P. A., et al. Perspectives in Melanoma: meeting report from the Melanoma Bridge (Dec. 1-3, 2022—Naples, Italy). J Transl Med 21, 508 (2023).
27. Kuczynski, E.A., et al. Impact of Chemical-Induced Mutational Load Increase on Immune Checkpoint Therapy in Poorly Responsive Murine Tumors. Mol Cancer Ther 17, 869-882 (2018).
28. Sharma, P., et al. The Next Decade of Immune Checkpoint Therapy. Cancer Discov 11, 838-857 (2021).
29. Bonaventura, P., et al. Cold Tumors: A Therapeutic Challenge for Immunotherapy. Front Immunol 10, 168 (2019).
30. Weber, J. S., et al. Individualised neoantigen therapy mRNA-4157 (V940) plus pembrolizumab versus pembrolizumab monotherapy in resected melanoma (KEYNOTE-942): a randomised, phase 2b study. The Lancet 403, 632-644 (2024).
31. Zhu, X., et al. Poly-ICLC promotes the infiltration of effector T cells into intracranial gliomas via induction of CXCL10 in IFN-alpha and IFN-gamma dependent manners. Cancer Immunol Immunother 59, 1401-1409 (2010).
32. Li, J., et al. Chemokine expression from oncolytic vaccinia virus enhances vaccine therapies of cancer. Mol Ther 19, 650-657 (2011).
33. Okada, H., et al. Induction of CD8+ T-cell responses against novel glioma-associated antigen peptides and clinical activity by vaccinations with alpha-type 1 polarized dendritic cells and polyinosinic-polycytidylic acid stabilized by lysine and carboxymethylcellulose in patients with recurrent malignant glioma. J Clin Oncol 29, 330-336 (2011).
34. Muthuswamy, R., et al. NF-kappaB hyperactivation in tumor tissues allows tumor-selective reprogramming of the chemokine microenvironment to enhance the recruitment of cytolytic T effector cells. Cancer Res 72, 3735-3743 (2012).
35. Muthuswamy, R., Wang, L., Pitteroff, J., Gingrich, J. R. & Kalinski, P. Combination of IFNalpha and poly-I: C reprograms bladder cancer microenvironment for enhanced CTL attraction. J Immunother Cancer 3, 6 (2015).
36. Muthuswamy, R., Corman, J. M., Dahl, K., Chatta, G. S. & Kalinski, P. Functional reprogramming of human prostate cancer to promote local attraction of effector CD8(+) T cells. Prostate 76, 1095-1105 (2016).
37. Francis, L., et al. Modulation of chemokines in the tumor microenvironment enhances oncolytic virotherapy for colorectal cancer. Oncotarget 7, 22174-22185 (2016).
38. Wong, J. L., Obermajer, N., Odunsi, K., Edwards, R. P. & Kalinski, P. Synergistic COX2 Induction by IFNgamma and TNFalpha Self-Limits Type-1 Immunity in the Human Tumor Microenvironment. Cancer Immunol Res 4, 303-311 (2016).
39. Muthuswamy, R., et al. Epinephrine promotes COX-2-dependent immune suppression in myeloid cells and cancer tissues. Brain Behav Immun 62, 78-86 (2017).
40. Obermajer, N., et al. Promoting the accumulation of tumor-specific T cells in tumor tissues by dendritic cell vaccines and chemokine-modulating agents. Nat Protoc 13, 335-357 (2018).
41. Theodoraki, M. N., et al. Helicase-Driven Activation of NFkappaB-COX2 Pathway Mediates the Immunosuppressive Component of dsRNA-Driven Inflammation in the Human Tumor Microenvironment. Cancer Res 78, 4292-4302 (2018).
42. Gandhi, S., et al. Systemic infusion of TLR 3-ligand and IFN-alpha in patients with breast cancer reprograms local tumor microenvironments for selective CTL influx. J Immunother Cancer 11(2023).
43. Nicodemus, C. F., Wang, L., Lucas, J., Varghese, B. & Berek, J. S. Toll-like receptor-3 as a target to enhance bioactivity of cancer immunotherapy. Am J Obstet Gynecol 202, 608 e601-608 (2010).
44. Mitchell, W. M. Efficacy of rintatolimod in the treatment of chronic fatigue syndrome/myalgic encephalomyelitis (CFS/ME). Expert Rev Clin Pharmacol 9, 755-770 (2016).
45. Jasani, B., Navabi, H. & Adams, M. Ampligen: a potential toll-like 3 receptor adjuvant for immunotherapy of cancer. Vaccine 27, 3401-3404 (2009).
46. Mailliard, R. B., et al. alpha-type-1 polarized dendritic cells: a novel immunization tool with optimized CTL-inducing activity. Cancer Res 64, 5934-5937 (2004).
47. Muthuswamy, R., et al. Ability of mature dendritic cells to interact with regulatory T cells is imprinted during maturation. Cancer Res 68, 5972-5978 (2008).
48. Kuai, R., et al. Elimination of established tumors with nanodisc-based combination chemoimmunotherapy. Sci Adv 4, eaao1736 (2018).
49. Mariathasan, S., et al. TGFbeta attenuates tumour response to PD-L1 blockade by contributing to exclusion of T cells. Nature 554, 544-548 (2018).
50. Shields, N. J., et al. Late-stage MC38 tumours recapitulate features of human colorectal cancer-implications for appropriate timepoint selection in preclinical studies. Front Immunol 14, 1152035 (2023).
51. Woo, S. R., et al. Immune inhibitory molecules LAG-3 and PD-1 synergistically regulate T-cell function to promote tumoral immune escape. Cancer Res 72, 917-927 (2012).
52. Fu, C. & Jiang, A. Dendritic Cells and CD8 T Cell Immunity in Tumor Microenvironment. Front Immunol 9, 3059 (2018).
53. Blunt, T., et al. Defective DNA-dependent protein kinase activity is linked to V(D)J recombination and DNA repair defects associated with the murine scid mutation. Cell 80, 813-823 (1995).
54. Shultz, L. D., et al. Human lymphoid and myeloid cell development in NOD/LtSz-scid IL2R gamma null mice engrafted with mobilized human hemopoietic stem cells. J Immunol 174, 6477-6489 (2005).
55. Hildner, K., et al. Batf3 deficiency reveals a critical role for CD8alpha+ dendritic cells in cytotoxic T cell immunity. Science 322, 1097-1100 (2008).
56. Edelson, B. T., et al. Peripheral CD103+ dendritic cells form a unified subset developmentally related to CD8alpha+ conventional dendritic cells. J Exp Med 207, 823-836 (2010).
57. Engelhardt, J. J., et al. Marginating dendritic cells of the tumor microenvironment cross-present tumor antigens and stably engage tumor-specific T cells. Cancer Cell 21, 402-417 (2012).
58. Aldinucci, D., Borghese, C. & Casagrande, N. The CCL5/CCR5 Axis in Cancer Progression. Cancers (Basel) 12(2020).
59. Tokunaga, R., et al. CXCL9, CXCL10, CXCL11/CXCR3 axis for immune activation—A target for novel cancer therapy. Cancer Treat Rev 63, 40-47 (2018). 60. Taylor, M. A., et al. Longitudinal immune characterization of syngeneic tumor models to enable model selection for immune oncology drug discovery. J Immunother Cancer 7, 328 (2019)
61. Shaheen, Z. R., et al. CCR5 is a required signaling receptor for macrophage expression of inflammatory genes in response to viral double-stranded RNA. Am J Physiol Regul Integr Comp Physiol 316, R525-R534 (2019).
62. Chang, L. Y., et al. Tumor-derived chemokine CCL5 enhances TGF-beta-mediated killing of CD8+T cells in colon cancer by T-regulatory cells. Cancer Res 72, 1092-1102 (2012).
63. Gonzalez-Martin, A., Gomez, L., Lustgarten, J., Mira, E. & Manes, S. Maximal T cell-mediated antitumor responses rely upon CCR5 expression in both CD4+ and CD8+T cells. Cancer Res 71, 5455-5466 (2011).
64. Hickman, H. D., et al. Chemokines control naive CD8+ T cell selection of optimal lymph node antigen presenting cells. J Exp Med 208, 2511-2524 (2011).
65. Rawat, K., et al. CCL5-producing migratory dendritic cells guide CCR5+ monocytes into the draining lymph nodes. J Exp Med 220(2023).

66. Spranger, S., Dai, D., Horton, B. & Gajewski, T. F. Tumor-Residing Batf3 Dendritic Cells Are Required for Effector T Cell Trafficking and Adoptive T Cell Therapy. Cancer Cell 31, 711-723 e714 (2017).
67. Chheda, Z. S., Sharma, R. K., Jala, V. R., Luster, A. D. & Haribabu, B. Chemoattractant Receptors BLT1 and CXCR3 Regulate Antitumor Immunity by Facilitating CD8+ T Cell Migration into Tumors. J Immunol 197, 2016-2026 (2016).
68. Hickman, H. D., et al. CXCR3 chemokine receptor enables local CD8+ T cell migration for the destruction of virus-infected cells. Immunity 42, 524-537 (2015).
69. Villarroel, V. A., Okiyama, N., Tsuji, G., Linton, J. T. & Katz, S. I. CXCR3-mediated skin homing of autoreactive CD8 T cells is a key determinant in murine graft-versus-host disease. J Invest Dermatol 134, 1552-1560 (2014).

1. A method for treating an individual for a programmed-death 1 (PD-1) immune checkpoint therapy resistant cancer, the method comprising administering to the individual a combination of a Toll-like receptor 3 (TLR3) agonist and interferon-alpha (IFNα), followed by administration of an anti-PD-1 immunotherapy.

2. The method of claim 1, wherein the TLR3 agonist and the IFNα are administered to the individual simultaneously and the anti-PD-1 immunotherapy is administered not more than 24 hours after administration of the TLR 3 agonist and the IFNα.

3. The method of claim 2, wherein the anti-PD-1 immunotherapy is administered not more than 12 hours after administration of the TLR 3 agonist and the IFNα.

4. The method of claim 2, wherein the anti-PD-1 immunotherapy is administered not more than 6 hours after administration of the TLR 3 agonist and the IFNα.

5. The method of claim 2, wherein the anti-PD-1 immunotherapy is administered not more than 3 hours after administration of the TLR 3 agonist and the IFNα.

6. The method of claim 2, wherein the anti-PD-1 immunotherapy is administered not more than 1 hour after administration of the TLR 3 agonist and the IFNα.

7. The method of claim 1, wherein the TLR3agonist is poly: IC or rintatolimod.

8. The method of claim 7, wherein the anti-PD-1 immunotherapy is pembrolizumab or nivolumab.

9. The method of claim 8, wherein the anti-PD-1 immunotherapy is the pembrolizumab.

10. The method of claim 9, wherein the pembrolizumab is administered to the individual not more than 1 hour after administration of rintatolimod and IFNα.
